(19) United States
(12) Patent Application Publication
(10) Pub. No.: US 2017/0298360 A1
(43) Pub. Date: Oct. 19, 2017

(54) LATS AND BREAST CANCER

(71) Applicants: Mohamed BENTIRES-ALJ, Kembs-Loechle (FR); Adrian BRITSCHGI, Hergiswil (CH)

(72) Inventors: Mohamed BENTIRES-ALJ, Kembs-Loechle (FR); Adrian BRITSCHGI, Hergiswil (CH)

(73) Assignee: FRIEDRICH MIESCHER INSTITUTE FOR BIOMEDICAL RESEARCH, Basel (US)

(21) Appl. No.: 15/513,935

(22) PCT Filed: Sep. 23, 2015
(86) PCT No.: PCT/IB2015/057338

§ 371 Date: Mar. 23, 2017

(30) Foreign Application Priority Data
Sep. 24, 2014 (EP) 14186104.7

(51) Int. Cl. C12N 15/113; A61K 31/138; A61K 39/395; C07K 16/40; A61K 31/713

(52) U.S. Cl. CPC C12N 15/1137; C07K 16/40; A61K 31/713; A61K 39/3955; A61K 31/138; C07K 2317/76; C12N 2310/14; C12N 2320/31

(57) ABSTRACT

The present invention relates to a method for treating breast cancer in a subject having a breast cancer of the estrogen receptor (ERα) negative type, which method comprises the step of administering to said subject a therapeutically effective amount of a modulator of the Large Tumor Suppressor Kinase (LATS). Also provided are a siRNA decreasing or silencing the expression of the Large Tumor Suppressor Kinase (LATS), and an antibody specifically binding to the Large Tumor Suppressor Kinase (LATS), for use to treat breast cancer of the estrogen receptor (ERα) negative type.

### FIELD OF THE INVENTION

The present invention relates to methods for treating breast cancer of the estrogen receptor negative type.

### BACKGROUND OF THE INVENTION

The mammary gland epithelium, consists of differentiated luminal epithelial and basal myoepithelial cells, as well as undifferentiated stem cells and more restricted progenitors. It is from this epithelium that breast cancer, the most common cancer in women, originates, yet the molecular mechanisms underlying breast epithelial hierarchy remain ill-defined and the interplay between the luminal and basal lineages has puzzled pathologists, developmental and cancer biologists for decades. Both lineages express a distinct set of keratins (and other markers) allowing their identification: mouse and human luminal cells express keratins (k)18, 8, 19 and/or estrogen (ER) and progesterone receptors (PR), and their basal counterparts express k5, 6 and/or 14 as well as p63 and/or α-smooth-muscle actin (α-SMA) (Howard and Gusterson, 2000; Petersen and Polyak, 2010; Visvader and Stingl, 2014). Loss of tumor suppressor genes, an initial step in transformation of normal cells (Hanahan and Weinberg, 2011), appears to be associated with deregulation of self-renewal and/or cell fate in many different organs such as liver, colon, prostate, brain and breast (Bienz and Clevers, 2000; Bouras et al., 2008; Cicalese et al., 2009; Li et al., 2002; Liu et al., 2008; Lu et al., 2013; Regad et al., 2009; Tschaharganeh et al., 2014; Yimlamai et al., 2014).

### SUMMARY OF THE INVENTION

In a study, using high-content confocal image-based shRNA screen for tumor suppressors regulating human breast cell fate, the present inventors have now surprisingly found that ablation of large tumor suppressor kinases 1 and 2 (LATS1/2), core members of the mammalian Hippo pathway (Halder and Johnson, 2011; Pan, 2010; Zhang et al., 2008; Zhao et al., 2010), a so-called tumor-suppressor pathway, enhances self-renewal and the number of progenitors and cells of the luminal lineage. They also further found that removal of LATS not only reprograms normal breast cells, but also renders estrogen receptor (ERα)-negative breast cancer cells, e.g. triple-negative breast cancer cells, ERα-positive and responsive to anti-hormone therapy, a therapy to which they were previously insensitive. The present inventors hence provide a new therapeutic approach for breast cancers of the estrogen receptor alpha (ERα) negative type, which method comprises the step of administering to the subject having said breast cancer a therapeutically effective amount of a modulator of the Large Tumor Suppressor Kinase (LATS). In some embodiments, the modulator inhibits the interaction between the estrogen receptor 1 and LATS and, in some cases, does not affect the kinase activity of LATS, in some cases it inhibits the interaction with a ubiquitin ligase complex. In some embodiments, the method of the invention further comprises the step of administering to said subject a therapeutically effective amount of an antagonist of the estrogen receptor after the step of administering to said subject a therapeutically effective amount of a modulator of LATS. In some embodiment, the antagonist of the estrogen receptor α is not fulvestrant. In some embodiments, the modulator is administered to the subject before, during or after radiation therapy, chemotherapy targeted therapy or after surgical removal of a primary tumor. In some embodiments the cancer is a solid tumor. In some embodiments, the modulator of LATS is an antibody or a siRNA.

The present invention also provides a siRNA decreasing or silencing the expression of the Large Tumor Suppressor Kinase (LATS), for use to treat breast cancer of the estrogen receptor (ERα) negative type.

The present invention also provides an antibody specifically binding to the Large Tumor Suppressor Kinase (LATS), for use to treat breast cancer of the estrogen receptor (ERα) negative type. In some embodiments, the antibody inhibits the interaction between the estrogen receptor α and LATS. In some embodiments, the antibody specifically binds to LATS, preferably to an epitope of LATS which is not the kinase domain of the enzyme.

The present invention also provides a combination for use to treat breast cancer of the estrogen receptor (ERα) negative type, which combination comprises (a) a modulator of the Large Tumor Suppressor Kinase (LATS) and (b) an antagonist of the estrogen receptor α, wherein the active ingredients are present in each case in free form or in the form of a pharmaceutically acceptable salt or any hydrate thereof, and optionally at least one pharmaceutically acceptable carrier; for simultaneous, separate or sequential use, wherein in case of sequential use, the a modulator of the Large Tumor Suppressor Kinase is administered first. In some embodiments, the modulator of the Large Tumor Suppressor Kinase (LATS) of this combination inhibits the interaction between the estrogen receptor α and LATS. In some embodiments of the combination the modulator of the Large Tumor Suppressor Kinase (LATS) is a siRNA. In some embodiments of the combination the antagonist of the estrogen receptor α is tamoxifen or other antihormone therapy. In some embodiments of the combination the modulator of the Large Tumor Suppressor Kinase (LATS) is a siRNA and the antagonist of the estrogen receptor is tamoxifen or other antihormone therapy.

The present invention also provides a method of screening for a modulator of LATS. In some embodiments, the hits of this screen are further screened in order to identify the hits influencing the kinase activity of LATS, wherein said hits are less preferred than the hits modulating LATS but not inhibiting its kinase activity.

### BRIEF DESCRIPTION OF THE DRAWINGS

The following drawings are illustrative of embodiments of the invention and are not meant to limit the scope of the invention as encompassed by the claims.

FIG. 1: Growth curves of SUM159 shNT and shLATS tumor-bearing mice treated with tamoxifen citrate (3 mg/kg). shLATS tumors are susceptible to anti-estrogen therapy. Data are means±SEM (n=5), **P<0.01.

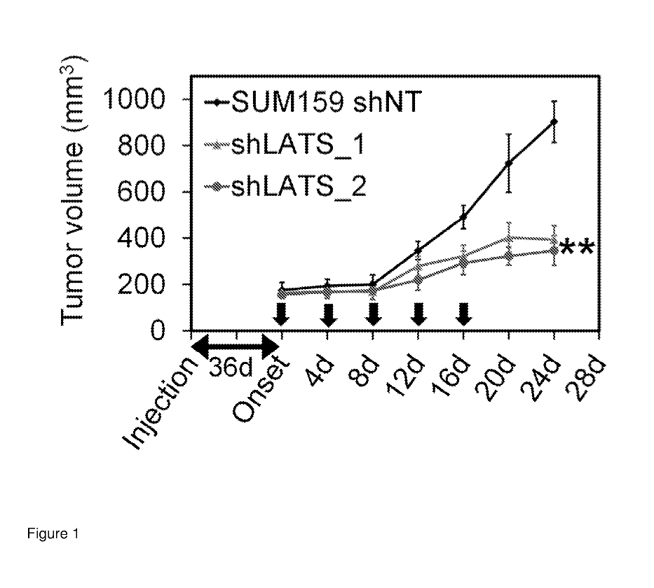

### DETAILED DESCRIPTION OF THE INVENTION

In a study, using high-content confocal image-based shRNA screen for tumor suppressors regulating human breast cell fate, the present inventors have now surprisingly found that ablation of large tumor suppressor kinases 1 and 2 (LATS1/2), core members of the mammalian Hippo pathway (Halder and Johnson, 2011; Pan, 2010; Zhang et al., 2008; Zhao et al., 2010), a so-called tumor-suppressor pathway, enhances self-renewal and the number of progenitors and cells of the luminal lineage. They also further found that removal of LATS not only reprograms normal breast cells, but also renders estrogen receptor (ERα)-negative breast cancer cells, e.g. triple-negative breast cancer cells, ERα-positive and responsive to anti-hormone therapy, a therapy to which they were previously insensitive. The present inventors hence provide a new therapeutic approach for breast cancers of the estrogen receptor (ERα) negative type, which method comprises the step of administering to the subject having said breast cancer a therapeutically effective amount of a modulator of the Large Tumor Suppressor Kinase (LATS). In some embodiments, the modulator inhibits the interaction between the estrogen receptor α and LATS and, in some cases, does not affect the kinase activity of LATS. In some embodiments, the method of the invention further comprises the step of administering to said subject a therapeutically effective amount of an antagonist of the estrogen receptor after the step of administering to said subject a therapeutically effective amount of a modulator of (LATS). In some embodiment, the antagonist of the estrogen receptor is not fulvestrant. In some embodiments, the modulator is administered to the subject before, during or after radiation therapy, chemotherapy targeted therapy or after surgical removal of a primary tumor. In some embodiments the cancer is a solid tumor. In some embodiments, the modulator of LATS is an antibody or a siRNA.

The present invention also provides a siRNA decreasing or silencing the expression of the Large Tumor Suppressor Kinase (LATS), for use to treat breast cancer of the estrogen receptor (ERα) negative type.

The present invention also provides an antibody specifically binding to the Large Tumor Suppressor Kinase (LATS), for use to treat breast cancer of the estrogen receptor (ERα) negative type. In some embodiments, the antibody inhibits the interaction between the estrogen receptor 1 and LATS. In some embodiments, the antibody specifically binds to LATS, preferably to an epitope of LATS which is not the kinase domain of the enzyme.

The present invention also provides a combination for use to treat breast cancer of the estrogen receptor (ERα) negative type, which combination comprises (a) a modulator of the Large Tumor Suppressor Kinase (LATS) and (b) an antagonist of the estrogen receptor, wherein the active ingredients are present in each case in free form or in the form of a pharmaceutically acceptable salt or any hydrate thereof, and optionally at least one pharmaceutically acceptable carrier; for simultaneous, separate or sequential use, wherein in case of sequential use, the a modulator of the Large Tumor Suppressor Kinase is administered first. In some embodiments, the modulator of the Large Tumor Suppressor Kinase (LATS) of this combination inhibits the interaction between the estrogen receptor α and LATS. In some embodiments of the combination the modulator of the Large Tumor Suppressor Kinase (LATS) is a siRNA. In some embodiments of the combination the antagonist of the estrogen receptor is tamoxifen. In some embodiments of the combination the modulator of the Large Tumor Suppressor Kinase (LATS) is a siRNA and the antagonist of the estrogen receptor is tamoxifen.

The present invention also provides a method of screening for a modulator of LATS. In some embodiments, the hits of this screen are further screened in order to identify the hits influencing the kinase activity of LATS, wherein said hits are less preferred than the hits modulating LATS but not inhibiting its kinase activity.

These and other aspects of the present invention should be apparent to those skilled in the art, from the teachings herein.

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single-and double-stranded DNA, DNA that is a mixture of single-and double-stranded regions, single-and double-stranded RNA, and RNA that is mixture of single-and double-stranded regions, hybrid molecules comprising DNA and RNA that may be single-stranded or, more typically, double-stranded or a mixture of single-and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SCC (750 mM NACl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6× SSPE (20× SSPE=3M NACl; 0.2M NaH2PO4; 0.02M EDTA, pH 7.4), 0.5% SDS, 30% formamide, 100 μg/ml salmon sperm blocking DNA; followed by washes at 50 degree C. with 1× SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments.

Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment”, “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al. , Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue. “Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 93%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity.

For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end.

The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 93%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=I, Window Size=sequence length, Gap Penalty—5, Gap Size Penalty—0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N-and C-terminal truncations of the subject sequence when calculating global percent identity. For subject sequences truncated at the N-and C-termini, relative to the query sequence, the percent identity is corrected by calculating the number of residues of the query sequence that are N-and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N-and C-termini of the subject sequence, which are not matched/aligned with the query sequence, are considered for the purposes of manually adjusting the percent identity score. That is, only query residue positions outside the farthest N-and C-terminal residues of the subject sequence. Only residue positions outside the N-and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem 268:22105-22111 (1993)) conducted extensive mutational analysis of human cytokine IL-1a.

They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N-or C-terminal residues of a protein retains such immunogenic activities can readily be determined by routine methods described herein and otherwise known in the art.

In one embodiment where one is assaying for the ability to bind or compete with full-length LATS polypeptide for binding to anti-LATS antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffasion assays, in situ immunoassays (using colloidal gold, enzyme or radioisotope labels, for example), western blots, precipitation reactions, agglutination assays (e.g., gel agglutination, assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Assays described herein and otherwise known in the art may routinely be applied to measure the ability of LATS polypeptides and fragments, variants derivatives and analogs thereof to elicit ERα expression (either in vitro or in vivo). For instance, an adhesion, protein binding, or reporter gene activation assay can be used.

The term “epitopes,” as used herein, refers to portions of a polypeptide having antigenic or immunogenic activity in an animal, in some embodiments, a mammal, for instance in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CHI, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e. g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Blochem., 270:3958-3964 (1995). Nucleic acids encoding the above epitopes can also be recombined with a gene of interest as an epitope tag (e.g., the hemagglutinin (“HA”) tag or flag tag) to aid in detection and punification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgGI, IgG2, IgG3, IgG4, IgAI and IgA2) or subclass of immunoglobulin molecule.

In addition, in the context of the present invention, the term “antibody” shall also encompass alternative molecules having the same function of specifically recognizing proteins, e.g. aptamers and/or CDRs grafted onto alternative peptidic or non-peptidic frames.

In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CHI, CH2, and CH3 domains.

Also included in the invention are antigen-binding fragments also comprising any combination of variable region(s) with a hinge region, CH1, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues.

Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide are also included in the present invention.

Antibodies may also be described or specified in terms of their binding affinity to a polypeptide Antibodies may act as agonists or antagonists of the recognized polypeptides. The invention also features receptor-specific antibodies which do not prevent ligand binding but prevent receptor activation. Receptor activation (i.e., signalling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387.

The antibodies as defined for the present invention include derivatives that are modified, i. e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immurnol. 13:65-93 (1995). For a detailed discussion of this technology for producing human antibodies and human monoclonal antibodies and protocols for producing such antibodies, see, e. g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity.

Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide). Further, in some embodiment of the invention an antibody, or fragment thereof, recognizing specifically LATS may be conjugated to a therapeutic moiety. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et aL, Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980.

The present invention is also directed to antibody-based therapies which involve administering antibodies of the invention to an animal, in some embodiments, a mammal, for example a human, patient to treat cancer. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

The invention also provides methods for ERα-negative breast cancer in a subject by administration to the subject of an effective amount of a LATS inhibitory compound or pharmaceutical composition comprising such inhibitory compound. In some embodiments, said inhibitory compound is an antibody or a siRNA. In a preferred aspect, the compound is substantially purified (e.g., substantially free from substances that limit its effect or produce undesired side-effects). The subject is in some embodiments, an animal, including but not limited to animals such as cows, pigs, horses, chickens, cats, dogs, camels, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e. g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g. , Goodson, in Medical Applications of Controlled Release, supra, vol. 2, pp. 115-138 (1984)). Other controlled release systems are discussed in the review by Langer (Science 249:1527-1533 (1990)).

The present invention also provides pharmaceutical compositions for use in the treatment of ER-negative breast cancer by inhibiting LATS. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, driied skim milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration. In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patients circumstances. Effective doses may be extrapolated from dose-response curves derived from in vitro or animal model test systems.

For antibodies, the dosage administered to a patient is typically 0.1 mg/kg to 100 mg/kg of the patients body weight. In some embodiments, the dosage administered to a patient is between 0.1 mg/kg and 20 mg/kg of the patient's body weight, for example 1 mg/kg to 10 mg/kg of the patients body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like.

The antibodies may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

By “biological sample” is intended any biological sample obtained from an individual, body fluid, cell line, tissue culture, or other source which contains the polypeptide of the present invention or mRNA.

As indicated, biological samples include body fluids (such as sputum, semen, lymph, sera, plasma, urine, synovial fluid and cerebro-spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

“RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown’ of gene expression. “siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid. The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA. Frequently, automatically generated alignments require manual alignment, exploiting the trained user's knowledge of the protein family being studied, e.g., biological knowledge of key conserved sites. One such alignment editor programs is Align (http://www.gwdg.de/dhepper/download/;Hepperle, D., 2001: Multicolor Sequence Alignment Editor. Institute of Freshwater Ecology and Inland Fisheries, 16775 Stechlin, Germany), although others, such as JalView or Cinema are also suitable. Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYL1P.

The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In a preferred embodiment, the inhibitor is a siRNA molecule and comprises between approximately 5 bp and 50 bp, in some embodiments, between 10 bp and 35 bp, or between 15 bp and 30 bp, for instance between 18 bp and 25 bp. In some embodiments, the siRNA molecule comprises more than 20 and less than 23 bp. Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides. The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among: a) a 3′ cap; b) a 5′ cap, c) a modified internucleoside linkage; or d) a modified sugar or base moiety. Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates. End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analysing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference. The siRNA molecule may be either synthesised de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918. The reaction may be carried out in solution or, in some embodiments, on solid phase or by using polymer supported reagents, followed by combining the synthesized RNA strands under conditions, wherein a siRNA molecule is formed, which is capable of mediating RNAi. It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used. For the avoidance of doubt, the term siRNA as used herein laso includes shRNA and shRNAmir. Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators). The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell. The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesise the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like). Alternatively a “naked” siRNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly. In a preferred embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product).

Inhibitors according to any embodiment of the present invention may be used in a monotherapy (e.g. use of siRNAs alone). However it will be appreciated that the inhibitors may be used as an adjunct, or in combination with other therapies.

The inhibitors of LATS may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site. The inhibitors of LATS may be used in a number of ways. For instance, systemic administration may be required in which case the compound may be contained within a composition that may, for example, be administered by injection into the blood stream.

Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate). The inhibitors of the invention may also be incorporated within a slow or delayed release device. Such devices may, for example, be inserted at the site of a tumor, and the molecule may be released over weeks or months. Such devices may be particularly advantageous when long term treatment with an inhibitor of LATS is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration. Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue. Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an inhibitor of LATS may be used for the treatment of cancer in the subject, depending upon which specific inhibitor is used. When the inhibitor is an siRNA molecule, the daily dose may be between 1 pg/kg of body weight and 100 mg/kg of body weight, in some embodiments, between approximately 10 pg/kg and 10 mg/kg, or between about 50 pg/kg and 1 mg/kg.

When the inhibitor (e.g. siNA) is delivered to a cell, daily doses may be given as a single administration (e.g. a single daily injection).

Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention.

Similarly, various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

The term “cancer” refers to a group of diseases in which cells are aggressive (grow and divide without respect to normal limits), invasive (invade and destroy adjacent tissues), and sometimes metastatic (spread to other locations in the body). These three malignant properties of cancers differentiate them from benign tumors, which are self-limited in their growth and do not invade or metastasize (although some benign tumor types are capable of becoming malignant).

“ER-negative breast cancer” refers to any breast cancer that does not overexpress the genes for estrogen receptor (ERα).

“Triple-negative breast cancer” refers to any breast cancer that does not overexpress the genes for estrogen receptor (ER), progesterone receptor (PR) or ErbB2/HER2/Neu. This subtype of breast cancer is clinically characterized as more aggressive and less responsive to standard treatment and associated with poorer overall patient survival. For the purpose of the present invention, triple-negative breast cancers include the basal-like and the claudin-low subtypes.

The “basal-like carcinoma” is a subtype of breast cancer defined by its gene expression and protein expression profile (Phenotypic evaluation of the basal-like subtype of invasive breast carcinoma, Modern Pathology 19:264-271 (2006)). In the current WHO breast tumor classification, these tumors are classified as ductal carcinoma. They are high grade, “triple-negative” tumors, i.e. they do not overexpress estrogen receptor, progesterone receptor or Her2/neu proteins.

Basal-like carcinomas tend to be more aggressive, with a poor prognosis. In the U.S., they are more frequent among black women, which may explain the higher mortality rate in this group.

The “claudin-low subtype”, or class, is a more recently described class that is often triple-negative but also has low cell-cell junction protein and has frequent lymphocytic infiltration.

As used herein, the term “metastasis” refers to the spread of cancer cells from one organ or body part to another area of the body, i.e. to the formation of metastases. This movement of tumor growth, i.e. metastasis or the formation of metastases, occurs as cancer cells break off the original tumor and spread e.g. by way of the blood or lymph system. Without wishing to be bound by theory, metastasis is an active process and involves an active breaking from the original tumor, for instance by protease digestion of membranes and or cellular matrices, transport to another site of the body, for instance in the blood circulation or in the lymphatic system, and active implantation at said other area of the body.

The present invention also provides a method of screening compounds to identify those which might be useful for treating cancer in a subject by modulating the expression or the protein levels of a LATS, as well as the so-identified compounds.

As used herein, “LATS”, also known as Large Tumor Suppressor Kinase, Large Tumor Suppressor, WARTS, EC.2.7.11.1 or EC.2.7.11, includes LATS1 and LATS2.

Serine/threonine-protein kinase LATS1 is an enzyme that in humans is encoded by the LATS1 gene. It has been associated with the Hippo signaling pathway. The protein encoded by this gene is a putative serine/threonine kinase that localizes to the mitotic apparatus and complexes with cell cycle controller CDC2 kinase in early mitosis. The protein is phosphorylated in a cell-cycle dependent manner, with late prophase phosphorylation remaining through metaphase. The N-terminal region of the protein binds CDC2 to form a complex showing reduced H1 histone kinase activity, indicating a role as a negative regulator of CDC2/cyclin A. In addition, the C-terminal kinase domain binds to its own N-terminal region, suggesting potential negative regulation through interference with complex formation via intramolecular binding. Biochemical and genetic data suggest a role as a tumor suppressor. This is supported by studies in knockout mice showing development of soft-tissue sarcomas, ovarian stromal cell tumors and a high sensitivity to carcinogenic treatments.

Serine/threonine-protein kinase LATS2 is an enzyme that in humans is encoded by the LATS2 gene. This gene encodes a serine/threonine protein kinase belonging to the LATS tumor suppressor family.

The protein localizes to centrosomes during interphase, and early and late metaphase. It interacts with the centrosomal proteins aurora-A and ajuba and is required for accumulation of gamma-tubulin and spindle formation at the onset of mitosis. It also interacts with a negative regulator of p53 and may function in a positive feedback loop with p53 that responds to cytoskeleton damage. Additionally, it can function as a co-repressor of androgen-responsive gene expression.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

Primary cell culture Breast epithelial cells were cultured in M5 medium (Duss et al., 2014) comprising 50% M199 medium (ANIMED/Bioconcept), 50% F12 (SIGMA) supplemented with 20 ng/ml EGF (PeproTech DE), 1× B-27 (Invitrogen/GIBCO), 1 nM 17-β-estradiol, 57 μM β-mercaptoethanol, 15 mM Hepes (SIGMA) and 1× penicillin/streptomycin (Invitrogen/GIBCO). M5 was used for all experiments with PHBECs. Uncoated tissue culture plastic (BD, Falcon Primaria) was used for monolayer colony formation and differentiation assays of PHBECs. From 500 to 2000 cells were seeded per well in 6-well plates and grown in M5 medium for 7-10 days prior to fixation or lysis. For 3D sphere formation assays, 2000-10000 cells/ml were grown in ultra-low attachment (ULA) dishes (Corning) for 6-10 days. All cells were cultured at 37° C. in a humidified 5% CO2, 5% O2 controlled incubator. For 3D breast cell fate screen, breast tissues were collected from women undergoing reduction mammoplasties, and handled and maintained according to protocols approved by The GNF Biomedical Institutional Review Board. To isolate single PHBECs, primary breast tissues were mechanically dissociated and digested in serum-free mammary epithelial growth media (MEGM, Lonza, Walkersville) containing 200-300 U/ml collagenase (Worthington), and then treated with 0.25% trypsin for 1-2 min and 1× red blood cell lysis buffer for 3 min, and filtered through cell strainers. The dissociated single PHBECs were cultured to generate mammospheres in modified serum-free MEGM media supplemented with 20 ng/ml bFGF (Invitrogen), 20 ng/ml EGF (Invitrogen), 0.5× B27 (Invitrogen), 4 μg/ml heparin (Sigma), 2 mM glutamine (Invitrogen), 5 μg/ml insulin (Sigma), 10⁻⁶ M hydrocortisone (StemCell Technologies). Bovine pituitary extract was excluded from MEGM kit.

3D breast cell fate screen PHBECs were plated at cell density of 3000 cells per well in a 384 well plate. 24 hours after seeding, the PHBECs were spin-transduced at 1800 RPM for one hour with lentivirus carrying a tumor suppressor shRNA library comprising 77 genes each represented by 3-5 different shRNAs. 24 h after transduction, the PHBECs were selected by 0.4 μg/ml puromycin. After 10 day culture (at day 13), the PHBEC-derived mammospheres were fixed by ice-cold MeOH for immunofluorescence staining and high-content confocal imaging. The anti-K14 (Abcam, LL002, IgG3) and anti-K19 (Abcam, A53-B/A2, IgG2a) were used as primary antibodies (at day 13), and Alexa Fluor 488-anti-mouse IgG3 and Alexa Fluor 633-anti-mouse IgG2a were used as secondary antibodies (at day 14), respectively. The automated “bottle valve” dispenser at GNF was used for media/reagent addition and removal via aspiration.

High-content confocal imaging and RSA analysis Mammospheres were examined in a 384 well plate by immunofluorescence staining and high-content confocal imaging using Opera High Content Screening System (PerkinElmer). The plate was imaged at a resolution of 20× and 3 optical sections separated by 30 μm each were collected. Optical sectioning was optimized to avoid collecting identical cells in different optical sections. In each well, 63 fields were collected for 3 different Z-section (total 189 images/well). A custom analysis using Acapella™ was performed on the images and the analysis is as follow: 1) assemble the different image plane (collate the different images into a single one); 2) detect cell nuclei using DAPI staining in each planes; 3) Detect cell boundaries using low level dapi fluorescence; 4) detect individual spheres as a group of cells in 3D (i.e., cells in different plans are associated to the containing sphere object); 5) Associate each from different plans cells to a given 3D sphere; 6) classify cells as K14 and/or K19 positive/negative into each sphere (and across each plans); 7) calculate and output sphere and cellular statistics (e.g., number, area, fraction of K14/K19 positive, etc.). In the downstream analysis, only spheres with more than 20 cells in total counted were kept for processing. The features extracted from the image data were then process using RSA analysis (Konig et al., 2007). In particular, the number of spheres and the fraction of K14/K19 double positive cells present in the spheres or in total cells were selected as biologically meaningful features. The genes were then ranked based on the RSA scores. RSA P values are calculated by the activity of the different shRNA and the reproducibility of these activities across multiple shRNAs available for each genes.

Lentiviral vectors, lentivirus production and infection For LATS knockdown, the following TRC pLKO clones (Sigma) were used: LATS_1=NM_004690.x-2666s1c1, LATS_2=NM_004690.x-3614s1c1, LATS_3=NM_014572.x-2894s1c1 and the MISSION® Non-Target shRNA Control Vector SHC002. For the competition experiment, GFP and ERα expressing lentiviral vectors were cloned as described in (Duss, BCR, 2007). Lentiviruses were produced by PEI transfection of 293 T cells as described (Britschgi et al., 2012; Duss et al., 2014). The titer of each lentiviral batch was determined on PHBECs or MCF10A cells. Cell lines were infected o/n in the presence of hexamethrine bromide (8 μg/mL). Infections were performed at a multiplicity of infection of 5-20 viral particles per cell. Selection with 1.25-1.5 μg/ml puromycin was applied 48 h after infection.

Animal experiments SCID/beige and SCID/NOD (Jackson Labs) were used in compliance with the Swiss laws for animal welfare and animal protocols were approved by the Swiss Cantonal Veterinary Office of Basel. For orthotopic engraftment of breast cancer cell lines, 2×10⁶ T47D or 1×10⁶ SUM159 cells were suspended in a 100 μl mixture of Basement Membrane Matrix Phenol Red-free (BD Biosciences) and PBS 1:1 and injected into the mouse mammary gland 4. For the estrogen-dependent T47D cell line, mice received estrogen-containing drinking water 2 days prior to injection. Tumor-bearing mice were randomized based on tumor volume prior to the initiation of treatment, which started when average tumor volume was at least 100 mm³. Fulvestrant and Tamoxifen hydrate dissolved in sunflower seed oil (all from Sigma) were injected intraperitoneally as indicated. Tumors were measured every 3 to 4 days with vernier calipers, and tumor volumes were calculated by the formula 0.5×(larger diameter)×(smaller diameter)².

Cell lines and in vitro experiments SUM159PT human breast cancer cells were from Asterand and propagated as recommended. All the other cell lines were from, and were cultured according to the protocol of ATCC. MCF10A cells were maintained as previously described (Bentires-Alj et al., 2006). For most experiments, MCF10A, SUM159 and MDA 436 cells were cultured in M5 medium and the medium was refreshed or replaced every 3 days. Antibody blocking experiments were performed by adding anti-AREG (R&D, MAB262, 0.5-10 μg/ml) or a mouse IgG antibody (R&D, 10 or 2.5 μg/ml) to the medium prior to seeding of the cells or transferring the conditioned medium. Cell viability was measured using the Cell Proliferation Reagent WST-1 (Roche). Colony formation assays were performed by seeding 500-1000 cells/well in 6-well plates. Cells were allowed to adhere overnight before treatment with inhibitors or solvent as indicated. Colonies were stained after 6-10 days with 0.2% crystal violet in PBS/4% formalin. Colony area was quantified using the Odyssey scanner and software (LICOR) or Image-J (Fiji 64 bit). For sphere formation assays, 4000 (passage 1) or 1000 (subsequent passages) MCF10A cells/ml were grown in ultra-low attachment (ULA) dishes (Corning) for 5-7 days. For LATS1 overexpression experiments, the following plasmids were used (addgene.org): LATS1 wt=p2×Flag CMV2 Lats1 (Plasmid #18971), LATS1 mut/kinase-truncated=p2×Flag CMV2 Lats1-N-mutant (Plasmid #18976) as well as the pFlag CMV2 empty vector control. Cells were transiently transfected using Lipofectamine2000 according to the manufacturer's protocol (Life Technologies).

Transient gene silencing procedures siRNAs were ordered as RP-HPLC purified duplexes, Silencer Select, from Invitrogen. The siRNA IDs were the following: siYAP s20366 and s20368, siTAZ s13807 and s13806, siERα s4823 and s4825, Silencer Select negative control siRNA No. 1 AM4611.

Transfections of siRNAs were performed according to the manufacture's guidelines (DharmaFect 1, Dharmacon).

Compounds and formulations Fulvestrant, 4-Hydroxytamoxifen (4-OHT), Tamoxifen (OHT), Cycloheximide and MG132 were from Sigma. Fulvestrant and MG-132 were prepared as 10 mMol/L stock solution in DMSO, 4-OHT and Cycloheximide as 10 mMol/L in ethanol and all stock solutions were stored at −20° C. For in vivo studies, fulvestrant and tamoxifen were dissolved in sunflower seed oil (Sigma) and injected intraperitoneal as indicated.

Flow cytometry For sorting of cells from normal breast tissue, organoids were dissociated with HyQtase (HyClone, Thermo Scientific) for 10 min at 37° C. and subsequent pipetting. Cells were filtered twice through 40-μm cell strainers (Falcon) to obtain single cells. 10⁶ cells were blocked in M5 medium for 10 min at 4° C. with antibodies against human CD16 (FcRIII, Clone 3G8, 1:50) and CD32 (FcRII, Clone FUN-2, 1:100), then washed and labeled in 100 μl M5 medium for 20 min at 4° C. with antibodies against human FITC-CD49f (Clone GoH3, 1:25), PerCP/Cy5.5-CD326 (EpCAM, Clone 9C4, 1:25), APC-cKIT (Clone 104D2, 1:20), PE-CD31 (Clone WM59, 1:33), PE-CD45 (Clone H130, 1:33), and PE-CD235ab (Clone HIR2, 1:33). DAPI (0.2%, Invitrogen) was added (1:250) 2 min before cell sorting.

FACS was carried out on BD FACSAria III (Becton Dickinson) using a 100 μm nozzle. Single cells were gated based on their forward and side scatter profiles and pulse-width was used to exclude doublets. Cells were gated based on their forward- and sideward-scatter. Dead cells (DAPI bright) and Lin+ cells (CD31+, CD45+ and CD235+) were gated out. For FACS of in vitro cultured cells and cell lines, cells were detached using Trypsin-EDTA (cell lines) or HyQTase (PHBECs), resuspended in growth medium and counted. For intracellular FACS analysis of ERα, cells were fixed and permeabilized using the BD Cytofix/Cytoperm kit according the manufacturer's protocol (BD Biosciences, Cat. No. 554714), then incubated with anti-ERα (Thermo/Fisher Scientific, clone SP1, 1:50) o/n at 4° C. in the dark followed by staining with a secondary anti-rabbit IgG-AlexaFluor488 (Biolegend) for 15 min at 4° C. in the dark prior to washing and analysis. ALDH1 assays was performed according to the manufacture's protocol (Stem Cells Technology). For analysis of surface markers, 10⁶ cells were incubated with APC-cKIT (Clone 104D2, 1:20), FITC-CD326/EpCAM (Clone 9C4, 1:25) or APC-CD10 (Clone HI10a, 1:20) for 20 min at 4° C. in the dark. All these antibodies were purchased from BioLegend. For Annexin V/propidium iodide staining, 0.5×10⁶ cells were washed with cold PBS/5% BSA, resuspended in 70 μl binding buffer and labelled with phycoerythrin (PE)-labelled antibody against Annexin V according to the manufacturer's protocol (Becton Dickinson). 10 min prior to analysis, propidium iodide (1 mg/ml, Sigma) was added at a final concentration of 1 μg/ml. For all experiments, at least 10⁴ cells per sample were analyzed with a FACScan flow cytometer BD FACSCalibur or BD LSR II SORP (Becton Dickinson).

Immunoblotting and immunoprecipitation Cells for immunoblotting and ELISA were lysed with RIPA buffer (50 mM Tris-HCl pH 8, 150 mM NACl, 1% NP-40, 0.5% sodium deoxycholate, 0.1% SDS), supplemented with 1× protease inhibitor cocktail (Complete Mini, Roche), 0.2 mMol/L sodium orthovanadate, 20 mM sodium fluoride and 1 mM phenylmethylsulfonyl fluoride. For ERα and LATS1 immunoprecipitations, cell lysates containing 500-1000 μg of protein were incubated with 1 μg of antibody and 20-50 μl of protein A-Sepharose beads (Zymed Laboratories, Inc., South San Francisco, Calif.) overnight at 4° C. Immunoprecipitates or whole cell lysates (30-80 μg) were subjected to SDS-PAGE, transferred to PVDF membranes (Immobilon-P, Millipore) and blocked for 1 h at room temperature with 5% milk in PBS-0.1% Tween 20. Membranes were then incubated overnight with antibodies as indicated and exposed to secondary HRP-coupled anti-mouse or -rabbit antibody at 1:5′000-10′000 for 1 h at room temperature. For each of the blots presented, the results shown are representative of at least three independent experiments. The following antibodies were used: LATS1(Cell Signaling, 1:1000), LATS2 (Abcam 1:500), ERK2 (Santa Cruz, 1:2000), pYAP (Ser127, Cell Signaling, 1:1000), YAP/TAZ (Cell Signaling, 1:1000), CCND1 (Santa Cruz, sc-718, 1:500), PCNA (Cell Signaling, 1:2000), c-KIT (Cell Signaling, 1:400), ERα (Thermo/Fisher Scientific, clone SP1, 1:500), keratin 18 (K18, Thermo Scientific, MS-142, 1:1000), keratin 14 (K14, Thermo Scientific, RB-9020, 1:2000), keratin 19 (K19, Thermo Scientific, MS-198, 1:1000), BMI-1 (Millipore, 05-637, 1:500), GFP (MBL, LabForceAG, 598), PARP (Cell Signaling, 1:1000), phosphor-ERα (Ser 118, Santa Cruz, sc-12915) and Mono/Poly-ubiquitin antibody (Enzo Life Sciences, clone FK2, BML-PW8810-0100, 1:1000).

Immunofluorescence, microscopy and image analysis Cells were fixed with ice-cold methanol/acetone (50/50 v/v) for 10 minutes at room temperature or with paraformaldehyde (4% in PBS) for 15 min at room temperature. The cells were then incubated at 4° C. overnight with the following primary antibodies: keratin 14 (RB-9020, 1:4,000), keratin 18 (MS-142, 1:2,000) or keratin 19 (MS-198 1:1,000) (Thermo Scientific). Goat anti-mouse, goat anti-rabbit or goat anti-guinea pig secondary antibodies coupled to Alexa 488, 568 or 633 (Molecular Probes, Invitrogen 1:500) were used for detection. For phase-contrast and fluorescence imaging of adherent cells, images were acquired with an inverted microscope (Nikon Eclipse Ti) using a 10× lens (Nikon Plan Fluor, NA 0.3) and a Nikon Ds-Fi camera. Immunofluorescent staining was analyzed with an inverted Zeiss Z1 microscope using a 20× air lens (Zeiss Plan-APOCHROME, NA 0.8) equipped with a motorized Zeiss scanning stage. Axiovision software was used to acquire and stitch images. Confocal pictures were taken with a LSM-510 confocal microscope (Zeiss) using a 20× air lens (Zeiss Plan-APOCHROME, NA 0.8). Both Zeiss microscopes were equipped with an Axio Cam MRC CCD (6.45 micron).

Percentages of covered area in each color channel (PHBEC colony formation) or percentages of positive cells (MCF10A colony formation) were quantified using Image-J (Fiji (64 bit)) software. Immunohistochemistry Tumor and normal tissue were fixed in 10% NBF (Neutral buffered formalin) for 24 h at 4° C., washed with 70%EtOH, embedded in paraffin, and 3 μm sections prepared and processed for hematoxyline and eosin staining and immunohistochemistory. Immunohistochemical staining was performed on formalin-fixed, paraffin-embedded tissue sections using a Bond-maX (Leica) fully automated system with anti-LATS1 (Sigma, HPA031804, 1:20).

Microarray Total RNA was extracted using the RNeasy Mini Kit according to the manufacturer's protocol (Qiagen) and reverse transcribed using 4 μM T7-(dT)24/T-7-(dN)6 primer mix (Affymetrix) and 150 units of Superscript II reverse transcriptase (Invitrogen). The synthesis of second-strand cDNA was performed by mixing 4 mM dNTPs, 6 units DNA polymerase I, and 0.4 units RNase H in a 20-μL reaction volume. cRNA was produced by in vitro transcription with a T7 RNA polymerase at 37° C. for 14 h using the MEGAscript T7 kit (Ambion) as per the manufacturer's instructions. For the second cycle, the first-strand cDNA was synthesized using 0.2 μg random primers from 9 μL of purified cRNA. The second-strand cDNA was produced using 10 μM T7-(dN)6 primer and 40 units DNA polymerase at 16° C. for 2 h, after which 10 units of T4 DNA polymerase (Invitrogen) was added and the incubation continued for another 10 min. The cDNA was in vitro transcribed with T7 RNA polymerase at 37° C. for 16 h. The single-strand cDNA was synthesized using 10 μg purified cRNA in the presence of 4 μg random primers, 0.2 M DTT, 12 mM dNTP+dUTP, and 750 units Superscript II (Roche Diagnostics) in a total volume of 20 μL. The cRNA was hydrolyzed with 2 units RNase H at 37° C. for 40 min. The sense cDNA was purified and eluted in 28 μL elution buffer. Amplified products were purified using the GeneChip cDNA Sample Cleanup Module (Affymetrix) with a 6,000-g centrifugation during the first two steps. To improve recovery from the columns, water or elution buffer was spun into the matrix at 50 g and left to stand for 4 min before a 16,000-g centrifugation. The quantity and purity of the cRNA and cDNA produced during the first and second rounds were evaluated using a NanoDrop ND-1000 spectrophotometer (Nanodrop Technologies). The cDNA was then fragmented by uracil DNA glycosylase and apurinic/apyrimidic endonuclease 1 and biotin-labeled with terminal deoxynucleotidyl transferase using the GeneChip WT Terminal labeling kit (Affymetrix).

Following hybridization, non-specifically bound nucleotides were removed by washing and specifically bound target detected using a GeneChip Hybridization, Wash and Stain kit and a GeneChip Fluidics Station 450 (Affymetrix). Hybridization was carried out with 5 μg of biotinylated target, which was incubated with a GeneChip human Gene 1.0 ST Array (Affymetrix) at 45° C. for 16 h. The arrays were scanned using a GeneChip Scanner 3000 7G (Affymetrix) and CEL files acquired using GeneChip Command Console Software (Affymetrix). Arrays were normalized and probeset-level expression values calculated with R/Bioconductor's (v2.14) ‘affy’ package using the rma function (www.bioconductor.org). Differential gene expression was determined using linear modeling implemented in the R/Bioconductor package ‘limma’. For general analysis, a cutoff of linear fold change >2 and P value of <0.01 were used. Gene set enrichment analysis (GSEA) was performed using the JAVA application from the Broad Institute v2.0 (http://www.broadinstitute.org.gsea).

Heatmaps show selected luminal progenitor, mature luminal and basal markers according to (Lim et al., 2010; Shehata et al., 2012; Skibinski et al., 2014). The microarray data from this publication are accessible through http://www.ncbi.nlm.nih.gov/geo/query/acc.cgi?acc= . . . .

Motif Activity Response Analysis (MARA) The MARA model (Balwierz et al., 2014) combines knowledge of gene expression levels (measured by microarray) with transcription factor binding sites to answer the question of which transcription factors are driving expression changes. Specifically, log expression levels of all genes present on the microarray were modeled as linear combinations of transcription factor activities. The coefficients of these combinations were determined by the number of transcription-factor binding sites in the proximal promoter regions. For each transcription-factor binding motif m and each sample (microarray) s, the activity Aₘₛ with the corresponding error, as well as significance of activity change of each binding motif was calculated.

RNA preparation and RQ-PCR Total RNA was extracted using the RNeasy Mini Kit according to the manufacturer's protocol (Qiagen). 1 μg of total RNA were transcribed using the Thermo Script RT-PCR System from Invitrogen. PCR and fluorescence detection were performed using the StepOnePlus Sequence Detection System (Applied Biosystems, Rotkreuz, Switzerland) according to the manufacturer's protocol in a reaction volume of 20 μl containing 1× TaqMan® Universal PCR Master Mix (Applied Biosystems) and 25 ng cDNA. The following probes were used: 1× Taqman® Gene Expression Assays (Applied Biosystems) for quantification of GAPDH (Hs02758991_g1), RPLP0 (Hs99999902_m1), CTGF (Hs01026927₋g1), Cyr61 (Hs00998500_g1), ESR1 (Hs01046818_m1), c-KIT (Hs00174029_m1), GREB1 (Hs00536409_m1), TFF1 (Hs00907239_m1), PGR (Hs01556702_m1), PRLR (Hs01061477_m1), AREG (Hs00950669_m1). Further, 1× IDT (Integrated DNA technologies) assays were used for quantification of: E-cadherin (Hs.PT.58.3324071), VIM (Hs.PT.58.38906895), N-cadherin (Hs.PT.58.26024443), fibronectin (FN) (Hs.PT.58.21141138), K18 (Hs.PT.58.38957843g), K19 (Hs.PT.58.3407073), ALDH1A3 (Hs.PT.56a.39201385), K14 (Hs.PT.58.39764207.g), CD10 (Hs.PT.58.38513876.g), ACTA2 (αSMA) (Hs.PT.56a.39662523). All measurements were performed in duplicates and the arithmetic mean of the Ct-values was used for calculations: target gene mean Ct-values were normalized to the respective housekeeping genes (GAPDH and RPLP0), mean Ct-values (internal reference gene, Ct), and then to the experimental control. Obtained values were exponentiated 2(−ΔΔCt) to be expressed as n-fold changes in regulation compared to the experimental control (2(−ΔΔCt) method of relative quantification (Livak and Schmittgen, 2001).

Statistical analysis. Each value reported represents the mean±S.E.M. of at least three independent experiments. Data were tested for normal distribution and Student's t-test, ANOVAs or nonparametric Mann-Whitney U/Wilcoxon-tests were applied. To account for multiple comparisons, Tukey HSD and Wilcoxon tests were performed. The programs JMP4 and JMP9 were used for all statistical tests (SAS, Cary, N.C., USA). P-values<0.05 were considered to be statistically significant.

Results

LATS depletion increases sphere formation and changes cell fate To assess the effects of tumor suppressor genes on self-renewal and cell fate of normal breast epithelial cells, freshly dissociated, unpassaged primary human breast epithelial cells (PHBECs) grown in non-adherent serum-free conditions were transduced with lentivirus carrying a tumor suppressor shRNA library comprising 77 genes each represented by 3-5 different shRNAs, selected with Puromycin, and cultured as mammospheres before immunofluorescent staining and high-content confocal imaging.

Mammospheres have been shown to be enriched for stem/progenitor cells endowed with high self-renewal and mammary reconstitution ability (Dontu et al., 2003; Liao et al., 2007). The inventors quantified number and size of mammospheres, as well as the fractions of basal K14 positive, luminal K19 positive and K14/K19 double positive progenitor cells in the mammospheres. Next, we applied redundant shRNA activity analysis (RSA), which allows computation of statistical scores that model the probability of a gene ‘hit’ based on the collective activities of multiple shRNAs per gene (Konig et al., 2007). The number of spheres and the fraction of K14/K19 double positive cells present in the spheres or in total cells were selected as biologically meaningful features. shRNAs targeting negative regulators of the mammalian Hippo pathway (LATS1, LATS2, FAT1, FAT3) scored high in increasing sphere formation and in enhancing the number of K14/K19 double positive cells. Individual shRNAs targeting LATS1, LATS2, FAT1 and FAT3 substantially increased the relative fractions of double positive and luminal K19-positive cells suggesting an effect of the Hippo pathway on breast cell fate.

To address the generality of LATS effects on cell fate and validate the results from this initial screen, the inventors generated PHBECs from three different donors, lentivirally transduced them with non-targeting control hairpins or shRNAs LATS_1 and LATS_2 targeting LATS1, and LATS_3 targeting both LATS1 and LATS2. They then performed mammosphere and colony formation assays to assess their self-renewal and differentiation potential, respectively. LATS knockdown increased self-renewal capacity and yielded densely clustered luminal and K14/K19 double positive colonies with an enhanced percentage of luminal and double positive cells and a decrease in basal cells.

Similarly, LATS knockdown in MCF10A cells, a spontaneously immortalized but untransformed breast epithelial cell line (Soule et al., 1990), resulted in enhanced self-renewal capacities, decreased fractions of K14-positive cells and increased number of K18-positive and K14/K18-positive cells.

Taken together, results from primary 3D culture screen as well as validation studies show that LATS is a critical regulator of self-renewal and cell fate in PHBECs.

LATS knockdown enriches for signatures of breast luminal progenitor and mature cells To characterize the effects of LATS knockdown on global gene expression and breast lineage profiles, the inventors performed microarrays on PHBECs derived from four different reduction mammoplasties in the presence or absence of LATS1 and/or LATS2. Principal component (PCA)-plot analysis revealed that samples lacking LATS clustered together, separated from controls. The majority of differentially regulated genes were common to both LATS shRNAs and no genes were significantly differentially expressed between shLATS_2 and shLATS_3 samples, even at low stringency cut-offs (P<0.05, fold change 2.0). 182 genes were differentially expressed at a cut-off of adjusted P-value <0.01, fold change >2.0 between shNT vs. shLATS_2 and shLATS_3 samples. Hierarchical clustering showed a high degree of inter-group separation in the presence or absence of LATS. Gene set enrichment analysis (GSEA) (Subramanian et al., 2005) revealed that the expression profile of PHBECs shLATS overlapped with previously published profiles from normal luminal breast progenitor as well as mature luminal cells (Lim et al., 2010), and was enriched for genes which are downregulated in normal basal breast cells. These data show that expression levels of basal lineage genes were reduced in shLATS PHBECs, while expression of luminal lineage genes was increased.

Next, the inventors addressed whether LATS removal affects the canonical Hippo pathway transcriptional co-activators YAP/TAZ (Badouel and McNeill, 2011). A decrease in pYAP-Ser127 (inhibitory site), an increase in YAP and TAZ protein levels and modest upregulation of the YAP/TAZ-downstream targets CTGF and Cyb61 were observed upon LATS knockdown. Surprisingly, despite this activation of YAP/TAZ signaling upon removal of LATS, we found no enrichment for YAP/TAZ-driven signatures when overlapping shLATS expression profile with previously published datasets (Cordenonsi et al., 2011; Matthews et al., 2009; Zhang et al., 2008).

To summarize, they found that ablation of LATS triggers a luminal signature in normal breast cells and despite activation of canonical YAP/TAZ signaling, cells lacking LATS were not imprinted by a global YAP/TAZ driven transcriptional profile.

LATS ablation upregulates ERα and imposes a luminal cell fate To identify the transcription factors that direct PHBECs lacking LATS towards the luminal fate, the inventors used a computational method (Integrated System for Motif Activity Response Analysis, ISMARA), which combines gene expression patterns with genome-wide predictions of transcription-factor-binding sites to model transcription factor activity (Balwierz et al., 2014). We found that activity of ERα was significantly higher in shLATS vs. shNT PHBECs. Consistently, ERα mRNA, the mRNA of c-KIT—a marker for luminal progenitors (Lim et al., 2009; Regan et al., 2012; Shehata et al., 2012)—, as well as mRNAs of several estrogen-targets were upregulated upon removal of LATS. LATS knockdown also increased luminal and decreased basal markers at the protein level. Further, shLATS PHBECs exhibited higher activity of transcription factors related to stem and progenitor cells (e.g., CEBPβ, SOX2), of downstream YAP/TAZ effectors (e.g., TEADs) and of factors related to epithelial-to-mesenchymal transition (e.g., ZEB1, SNAIL).

To assess effects of LATS removal on the cellular hierarchy, the inventors subjected shNT and shLATS PHBECs to flow cytometry analysis. Fractions of c-KIT positive cells as well as fractions of cells with high ALDH1 activity, which—similar to c-KIT—has been shown to be associated with luminal progenitors (Eirew et al., 2012), were significantly increased upon removal of LATS. At the same time, shLATS PHBECs exhibited higher expression of the luminal epithelial cell surface marker EpCAM and slightly lower expression of the basal marker CD10. Intracellular flow cytometry analysis of estrogen receptor expression combined with surface staining for c-KIT showed a 5.5, 4.6 and 8.5-fold increase in the percentage of c-KIT, ERα and ERα/c-KIT positive cells in the absence of LATS.

They then assed the effect of LATS knockdown on MCF10A cells which exhibit a predominantly basal cell-like phenotype with a low level of expression of luminal markers (Neve et al., 2006; Soule et al., 1990). MCF10A cells lacking LATS and cultured for at least 4 days in M5 medium adopted a more luminal-epithelial, cobblestone-like morphology and formed smaller colonies as compared to control cells. In line with this observation, the epithelial marker e-cadherin was significantly upregulated in shLATS cells, while mesenchymal markers remained unchanged (vimentin, n-cadherin) or were downregulated (fibronectin). Markers of mature luminal and luminal progenitor cells increased dramatically and as for PHBECs, YAP/TAZ signaling was modestly activated.

These data show that removal of LATS increases ERα activity and triggers normal breast epithelial cells to acquire a mature luminal and luminal progenitor fate.

LATS ablation favors breast luminal cell survival YAP and TAZ were shown to promote cell proliferation and survival in epithelial cells (Pan, 2010; Varelas, 2014) and LATS mediated downregulation of YAP is required for anoikis in non-transformed MCF10A cells (Zhao et al., 2012). Consistently, LATS knockdown in breast epithelial cells increased their proliferation as evidenced by an increase in CCND1 and PCNA protein levels, an increase in cell number and an increase in the number of colonies upon removal of LATS. In suspension, LATS knockdown cells also had higher levels of YAP/TAZ and decreased anoikis compared with control cells. Given the previous findings that LATS ablation increased luminal cell fate, these data raised the possibility LATS removal confers a survival advantage specifically to breast luminal cells. To test this hypothesis, the inventors assessed the effects of LATS knockdown in different subpopulations of PHBECs. They used FACS to sort different human breast lineages (Lim et al., 2009; Smart et al., 2011) confirmed purity of the subpopulations by RQ-PCR, and infected cells with control or LATS shRNA expressing vectors. LATS ablation significantly enhanced colony and sphere formation capacity specifically in the luminal cell lineage.

They then modulated ERα expression in shNT and shLATS MCF10A cells which almost entirely lack expression of ERα (Pilat et al., 1996). They stably infected shNT and shLATS MCF10A cells with GFP or GFP and ERα and mixed small fractions (2-3%) thereof with a bulk of MCF10A shNT cells. In the setting of a competition experiment, they cultured the cells in suspension and assessed the number of sphere-forming GFP-positive cells. The fraction of ERα -infected non-targeting control cells did not increase, but we observed a slight increase in the fraction of shLATS-GFP cells and a substantial, 4.5 fold increase in the fraction of shLATS-ERα cells. Taken together, these data show a subpopulation specific effect of loss of LATS in breast epithelial cells.

YAP/TAZ and ERα—signaling promote sphere formation and breast luminal fate To assess the relative contribution of YAP/TAZ and ERα signaling to the enhanced sphere formation capacity and the increase in luminal markers of shLATS breast epithelial cells, the inventors inhibited endogeneous levels of YAP, TAZ and ERα in MCF10A shNT and MCF10A shLATS cells using siRNA. While sphere formation capacity was almost fully reverted upon siYAP/TAZ, this knockdown partially reduced the induction of luminal markers in shLATS cells. YAP/TAZ also contributed to the competitive advantage of ER positive cells lacking LATS. Depletion of ERα, on the other hand, fully abrogated the upregulation of luminal markers, but only partially reverted the enhanced sphere formation capacity in shLATS cells. Ablation of both YAP/TAZ and ERα signaling in shLATS cells, fully reversed the increase in luminal markers and sphere formation capacity in cells lacking LATS. These data show that both YAP/TAZ and ERα contribute to sphere formation and direct towards the luminal fate, albeit not to the same extent.

Next, the inventors searched the literature for potential downstream effectors of ER and YAP/TAZ signaling. The EGF-growth factor-like ligand amphiregulin (AREG) has crucial effects in mouse mammary gland development (Brisken and O'Malley, 2010; McBryan et al., 2008) and was shown to be regulated by both ERα (McBryan et al., 2008) and YAP/TAZ (Yang et al., 2012; Zhang et al., 2009).

They assessed whether AREG was the downstream effector of ERα and YAP/TAZ in enhancing sphere formation in cells lacking LATS. AREG expression increased in shLATS MCF10A cells grown in M5 medium as spheres, and depletion of both YAP/TAZ and ERα reverted increased AREG mRNA levels of shLATS cells back to the levels of shNT control cells. AREG inhibition by a blocking antibody dramatically decreased sphere formation capacity in the absence of LATS compared with controls and conditioned media from LATS-knockdown cultures significantly enhanced the numbers of spheres formed by MCF10A cells, an effect that was blocked by adding an AREG blocking antibody. These data suggest amphiregulin as the downstream effector of ERα and YAP/TAZ in inducing sphere formation in the absence of LATS.

LATS targets ERα to proteasomal degradation The pronounced upregulation of ERα protein upon removal of LATS compared with a modest increase in ERα mRNA prompted the inventors to address if the LATS kinases would interact directly with ERα, influencing its posttranslational processing or stability. Knockdown of LATS in the ER-positive breast cancer line T47D enhanced levels of ERα and phospho-ERα, as well as expression of estrogen target genes. Immunoprecipitation experiment showed that LATS1 and ERα physically interact and expression of full-length LATS1 or LATS1 lacking the kinase domain in cells with low endogenous LATS decreased ERα levels in several ERα-positive breast cancer cell lines, suggesting that the kinase activity of LATS1 was dispensable for ERα regulation. Inhibition of translation using Cycloheximide showed that that LATS knockdown increased the half-life of ERα upon stimulation with estrogen by 6-fold. Since proteasomal degradation is a crucial mechanism for regulation of ERα protein levels (Lee and Lee, 2012), the inventors treated LATS1 overexpressing cells with the proteasome inhibitor MG132 and found that it prevented LATS1-induced degradation of ERα. Ubiquitination of ERα precedes its proteasomal degradation, and we found that in the absence of LATS, ubiquitination of ERα is reduced, providing an explanation for its stabilization and accumulation. These data show that LATS directly interacts with ERα and that it facilitates its proteasomal degradation via ubiquitination.

Expression of LATS1 is reduced in breast cancers with dismal prognosis Expression profile of PHBECs lacking LATS was significantly enriched in genes associated with ER-positive and luminal breast cancers (Charafe-Jauffret et al., 2006; Doane et al., 2006). In the normal breast, LATS1 was expressed in the basal layer but not in luminal cells, in line with our observation that ablation of LATS promotes a luminal fate. The inventors then assessed LATS1 protein expression levels in primary human breast tumors and cancer cell lines, both by immunoblotting and by IHC and found that LATS1 was almost absent in all tumor samples, lowest in tumors which expressed high levels of ERα, and reduced or undetectable in 7 out of 9 basal and 6 out of 6 luminal breast cancer cell lines compared with MCF10A cells. Notably, within the same patient sample, LATS1 levels gradually decreased when progressing from normal breast structures to hyperplastic ducts to ductal-carcinoma in situ-like structures. Analysis of publically available datasets (Gyorffy et al., 2012) revealed that low expression of LATS1 mRNA was associated with reduced relapse-free survival of breast cancer patients.

Loss of LATS1 reduces sensitivity to fulvestrant Next, the inventors wanted to test if loss of LATS affects proliferation, tumor growth and treatment response in ER-positive breast cancer models. For the luminal breast cancer subtype, anti-estrogen treatment fulvestrant or tamoxifen are the most commonly used (Howell, 2006; Ignatiadis and Sotiriou, 2013). Downregulation of LATS in T47D and MCF7 cells reduced sensitivity to the estrogen-down-regulator fulvestrant but had no effect on the viability of untreated or tamoxifen treated cells. Consistently, fulvestrant failed to degrade ERα and to reduce tumor growth of cancer cells grown as xenograft. They conclude that loss of LATS in luminal breast cancer models stabilizes ERα and confers resistance to treatments targeting the downregulation of ERα.

Ablation of LATS renders triple-negative breast cancer cells ERα-positive and responsive to anti-estrogen therapy Because knockdown of LATS promoted luminal fate and ER expression in non-transformed breast cells, the inventors asked whether it also does in basal breast cancer models. Ablation of LATS in in triple-negative lines SUM159 and MDA 436 cultured in M5 medium triggered expression of ERα. Viability of cells with and without LATS was similar, but treatment with tamoxifen reduced colony formation in cells lacking LATS compared to controls while treatment with fulvestrant had no effect. These findings are consistent with a restoration of estrogen dependency and of the failure of fulvestrant to degrade ERα in cancer cells without LATS. This developed sensitivity of basal cells to tamoxifen treatment was also observed in vivo, where SUM159 shLATS xenografts stopped growing when tumor-bearing mice were treated with tamoxifen. These data show that in basal, ER-negative cells, stabilization of ERα protein upon inhibition of LATS rendered them sensitive to tamoxifen treatment.

Artavanis-Tsakonas, S., Rand, M. D., and Lake, R. J. (1999). Notch signaling: cell fate control and signal integration in development. Science 284, 770-776.

Badouel, C., and McNeill, H. (2011). SnapShot: The hippo signaling pathway. Cell 145, 484-484 e481.

Bhat, K. P., Salazar, K. L., Balasubramaniyan, V., Wani, K., Heathcock, L., Hollingsworth, F., James, J. D., Gumin, J., Diefes, K. L., Kim, S. H., et al. (2011). The transcriptional coactivator TAZ regulates mesenchymal differentiation in malignant glioma. Genes & development 25, 2594-2609.

Bienz, M., and Clevers, H. (2000). Linking colorectal cancer to Wnt signaling. Cell 103, 311-320.

Bissell, M. J., and Labarge, M. A. (2005). Context, tissue plasticity, and cancer: are tumor stem cells also regulated by the microenvironment? Cancer cell 7, 17-23.

Blanpain, C., and Fuchs, E. (2014). Stem cell plasticity. Plasticity of epithelial stem cells in tissue regeneration. Science 344, 1242281.

Booth, B. W., Boulanger, C. A., Anderson, L. H., Jimenez-Rojo, L., Brisken, C., and Smith, G. H. (2010).

Amphiregulin mediates self-renewal in an immortal mammary epithelial cell line with stem cell characteristics. Experimental cell research 316, 422-432.

Bouras, T., Pal, B., Vaillant, F., Harburg, G., Asselin-Labat, M. L., Oakes, S. R., Lindeman, G. J., and Visvader, J. E. (2008). Notch signaling regulates mammary stem cell function and luminal cell-fate commitment. Cell stem cell 3, 429-441.

Brisken, C., and Duss, S. (2007). Stem cells and the stem cell niche in the breast: an integrated hormonal and developmental perspective. Stem cell reviews 3, 147-156.

Brisken, C., and O'Malley, B. (2010). Hormone action in the mammary gland. Cold Spring Harbor perspectives in biology 2, a003178.

Britschgi, A., Andraos, R., Brinkhaus, H., Klebba, I., Romanet, V., Muller, U., Murakami, M., Radimerski, T., and Bentires-Alj, M. (2012). JAK2/STATS inhibition circumvents resistance to P13K/mTOR blockade: a rationale for cotargeting these pathways in metastatic breast cancer. Cancer cell 22, 796-811.

Cantone, I., and Fisher, A. G. (2013). Epigenetic programming and reprogramming during development. Nature structural & molecular biology 20, 282-289.

Charafe-Jauffret, E., Ginestier, C., Monville, F., Finetti, P., Adelaide, J., Cervera, N., Fekairi, S., Xerri, L., Jacquemier, J., Birnbaum, D., et al. (2006). Gene expression profiling of breast cell lines identifies potential new basal markers. Oncogene 25, 2273-2284.

Chen, D., Sun, Y., Wei, Y., Zhang, P., Rezaeian, A. H., Teruya-Feldstein, J., Gupta, S., Liang, H., Lin, H. K., Hung, M. C., et al. (2012). LIFR is a breast cancer metastasis suppressor upstream of the Hippo-YAP pathway and a prognostic marker. Nature medicine 18, 1511-1517.

Chen, Q., Zhang, N., Gray, R. S., Li, H., Ewald, A. J., Zahnow, C. A., and Pan, D. (2014). A temporal requirement for Hippo signaling in mammary gland differentiation, growth, and tumorigenesis. Genes & development 28, 432-437.

Chu, I., Arnaout, A., Loiseau, S., Sun, J., Seth, A., McMahon, C., Chun, K., Hennessy, B., Mills, G. B., Nawaz, Z., et al. (2007). Src promotes estrogen-dependent estrogen receptor alpha proteolysis in human breast cancer. The Journal of clinical investigation 117, 2205-2215.

Cicalese, A., Bonizzi, G., Pasi, C. E., Faretta, M., Ronzoni, S., Giulini, B., Brisken, C., Minucci, S., Di Fiore, P. P., and Pelicci, P. G. (2009). The tumor suppressor p53 regulates polarity of self-renewing divisions in mammary stem cells. Cell 138, 1083-1095.

Cordenonsi, M., Zanconato, F., Azzolin, L., Forcato, M., Rosato, A., Frasson, C., lnui, M., Montagner, M., Parenti, A. R., Poletti, A., et al. (2011). The Hippo transducer TAZ confers cancer stem cell-related traits on breast cancer cells. Cell 147, 759-772.

Couse, J. F., and Korach, K. S. (1999). Estrogen receptor null mice: what have we learned and where will they lead us? Endocrine reviews 20, 358-417.

Derynck, R., and Akhurst, R. J. (2007). Differentiation plasticity regulated by TGF-beta family proteins in development and disease. Nature cell biology 9, 1000-1004.

Dhananjayan, S. C., Ramamoorthy, S., Khan, O. Y., Ismail, A., Sun, J., Slingerland, J., O'Malley, B. W., and Nawaz, Z. (2006). WW domain binding protein-2, an E6-associated protein interacting protein, acts as a coactivator of estrogen and progesterone receptors. Molecular endocrinology 20, 2343-2354.

Diepenbruck, M., Waldmeier, L., Ivanek, R., Berninger, P., Arnold, P., van Nimwegen, E., and Christofori, G. (2014). Tead2 expression levels control the subcellular distribution of Yap and Taz, zyxin expression and epithelial-mesenchymal transition. Journal of cell science 127, 1523-1536.

Doane, A. S., Danso, M., Lal, P., Donaton, M., Zhang, L., Hudis, C., and Gerald, W. L. (2006). An estrogen receptor-negative breast cancer subset characterized by a hormonally regulated transcriptional program and response to androgen. Oncogene 25, 3994-4008.

Dontu, G., Abdallah, W. M., Foley, J. M., Jackson, K. W., Clarke, M. F., Kawamura, M. J., and Wicha, M. S. (2003). In vitro propagation and transcriptional profiling of human mammary stem/progenitor cells. Genes & development 17, 1253-1270.

Dupont, J., Renou, J. P., Shani, M., Hennighausen, L., and LeRoith, D. (2002). PTEN overexpression suppresses proliferation and differentiation and enhances apoptosis of the mouse mammary epithelium. The Journal of clinical investigation 110, 815-825.

Duss, S., Brinkhaus, H., Britschgi, A., Cabuy, E., Frey, D. M., Schaefer, D. J., and Bentires-Alj, M. (2014). Mesenchymal precursor cells maintain the differentiation and proliferation potentials of breast epithelial cells. Breast cancer research: BCR 16, R60.

Eirew, P., Kannan, N., Knapp, D. J., Vaillant, F., Emerman, J. T., Lindeman, G. J., Visvader, J. E., and Eaves, C. J. (2012). Aldehyde dehydrogenase activity is a biomarker of primitive normal human mammary luminal cells. Stem cells 30, 344-348.

Fernandez-Valdivia, R., and Lydon, J. P. (2012). From the ranks of mammary progesterone mediators, RANKL takes the spotlight. Molecular and cellular endocrinology 357, 91-100.

Fillmore, C. M., Gupta, P. B., Rudnick, J. A., Caballero, S., Keller, P. J., Lander, E. S., and Kuperwasser, C. (2010). Estrogen expands breast cancer stem-like cells through paracrine FGF/Tbx3 signaling. Proc Natl Acad Sci USA 107, 21737-21742.

Friedmann-Morvinski, D., Bushong, E. A., Ke, E., Soda, Y., Marumoto, T., Singer, O., Ellisman, M. H., and Verma, I. M. (2012). Dedifferentiation of neurons and astrocytes by oncogenes can induce gliomas in mice. Science 338, 1080-1084.

Garbe, J. C., Pepin, F., Pelissier, F. A., Sputova, K., Fridriksdottir, A. J., Guo, D. E., Villadsen, R., Park, M., Petersen, O. W., Borowsky, A. D., et al. (2012). Accumulation of multipotent progenitors with a basal differentiation bias during aging of human mammary epithelia. Cancer research 72, 3687-3701.

Gyorffy, B., Lanczky, A., and Szallasi, Z. (2012). Implementing an online tool for genome-wide validation of survival-associated biomarkers in ovarian-cancer using microarray data from 1287 patients. Endocrine-related cancer 19, 197-208.

Halder, G., and Johnson, R. L. (2011). Hippo signaling: growth control and beyond. Development 138, 9-22.

Hanahan, D., and Weinberg, R. A. (2011). Hallmarks of cancer: the next generation. Cell 144, 646-674.

Harvey, K. F., Zhang, X., and Thomas, D. M. (2013). The Hippo pathway and human cancer. Nature reviews Cancer 13, 246-257.

Howard, B. A., and Gusterson, B. A. (2000). Human breast development. Journal of mammary gland biology and neoplasia 5, 119-137.

Howell, A. (2006). Pure oestrogen antagonists for the treatment of advanced breast cancer. Endocrine-related cancer 13, 689-706.

Iglesias-Bartolome, R., and Gutkind, J. S. (2011). Signaling circuitries controlling stem cell fate: to be or not to be. Current opinion in cell biology 23, 716-723.

Ignatiadis, M., and Sotiriou, C. (2013). Luminal breast cancer: from biology to treatment. Nature reviews Clinical oncology 10, 494-506.

Kalyuga, M., Gallego-Ortega, D., Lee, H. J., Roden, D. L., Cowley, M. J., Caldon, C. E., Stone, A., Allerdice, S. L., Valdes-Mora, F., Launchbury, R., et al. (2012). ELFS suppresses estrogen sensitivity and underpins the acquisition of antiestrogen resistance in luminal breast cancer. PLoS biology 10, e1001461.

Kannan, N., Huda, N., Tu, L., Droumeva, R., Aubert, G., Chavez, E., Brinkman, R. R., Lansdorp, P., Emerman, J., Abe, S., et al. (2013). The luminal progenitor compartment of the normal human mammary gland constitutes a unique site of telomere dysfunction. Stem cell reports 1, 28-37.

Katoh, M. (2012). Function and cancer genomics of FAT family genes (review). International journal of oncology 41, 1913-1918.

Konig, R., Chiang, C. Y., Tu, B. P., Yan, S. F., DeJesus, P. D., Romero, A., Bergauer, T., Orth, A., Krueger, U., Zhou, Y., et al. (2007). A probability-based approach for the analysis of large-scale RNAi screens. Nature methods 4, 847-849.

LaBarge, M. A., Petersen, O. W., and Bissell, M. J. (2007). Of microenvironments and mammary stem cells. Stem cell reviews 3, 137-146.

Lamar, J. M., Stern, P., Liu, H., Schindler, J. W., Jiang, Z. G., and Hynes, R. O. (2012). The Hippo pathway target, YAP, promotes metastasis through its TEAD-interaction domain. Proc Natl Acad Sci USA 109, E2441-2450.

Lau, A. N., Curtis, S. J., Fillmore, C. M., Rowbotham, S. P., Mohseni, M., Wagner, D. E., Beede, A. M., Montoro, D. T., Sinkevicius, K. W., Walton, Z. E., et al. (2014). Tumor-propagating cells and Yap/Taz activity contribute to lung tumor progression and metastasis. The EMBO journal 33, 468-481.

Lee, J. H., and Lee, M. J. (2012). Emerging roles of the ubiquitin-proteasome system in the steroid receptor signaling. Archives of pharmacal research 35, 397-407.

Lei, Q. Y., Zhang, H., Zhao, B., Zha, Z. Y., Bai, F., Pei, X. H., Zhao, S., Xiong, Y., and Guan, K. L. (2008). TAZ promotes cell proliferation and epithelial-mesenchymal transition and is inhibited by the hippo pathway. Mol Cell Biol 28, 2426-2436.

Li, G., Robinson, G. W., Lesche, R., Martinez-Diaz, H., Jiang, Z., Rozengurt, N., Wagner, K. U., Wu, D. C., Lane, T. F., Liu, X., et al. (2002). Conditional loss of PTEN leads to precocious development and neoplasia in the mammary gland. Development 129, 4159-4170.

Lian, I., Kim, J., Okazawa, H., Zhao, J., Zhao, B., Yu, J., Chinnaiyan, A., Israel, M. A., Goldstein, L. S., Abujarour, R., et al. (2010). The role of YAP transcription coactivator in regulating stem cell self-renewal and differentiation. Genes & development 24, 1106-1118.

Liao, M. J., Zhang, C. C., Zhou, B., Zimonjic, D. B., Mani, S. A., Kaba, M., Gifford, A., Reinhardt, F., Popescu, N. C., Guo, W., et al. (2007). Enrichment of a population of mammary gland cells that form mammospheres and have in vivo repopulating activity. Cancer research 67, 8131-8138.

Lim, E., Vaillant, F., Wu, D., Forrest, N. C., Pal, B., Hart, A. H., Asselin-Labat, M. L., Gyorki, D. E., Ward, T., Partanen, A., et al. (2009). Aberrant luminal progenitors as the candidate target population for basal tumor development in BRCA1 mutation carriers. Nature medicine 15, 907-913.

Lim, E., Wu, D., Pal, B., Bouras, T., Asselin-Labat, M. L., Vaillant, F., Yagita, H., Lindeman, G. J., Smyth, G. K., and Visvader, J. E. (2010). Transcriptome analyses of mouse and human mammary cell subpopulations reveal multiple conserved genes and pathways. Breast cancer research: BCR 12, R21.

Liu, S., Ginestier, C., Charafe-Jauffret, E., Foco, H., Kleer, C. G., Merajver, S.D., Dontu, G., and Wicha, M. S. (2008). BRCA1 regulates human mammary stem/progenitor cell fate. Proc Natl Acad Sci USA 105, 1680-1685.

Lu, T. L., Huang, Y. F., You, L. R., Chao, N. C., Su, F. Y., Chang, J. L., and Chen, C. M. (2013). Conditionally ablated Pten in prostate basal cells promotes basal-to-luminal differentiation and causes invasive prostate cancer in mice. The American journal of pathology 182, 975-991.

Matthews, L., Gopinath, G., Gillespie, M., Caudy, M., Croft, D., de Bono, B., Garapati, P., Hemish, J., Hermjakob, H., Jassal, B., et al. (2009). Reactome knowledgebase of human biological pathways and processes. Nucleic acids research 37, D619-622.

McBryan, J., Howlin, J., Napoletano, S., and Martin, F. (2008). Amphiregulin: role in mammary gland development and breast cancer. Journal of mammary gland biology and neoplasia 13, 159-169.

Meier-Abt, F., Milani, E., Roloff, T., Brinkhaus, H., Duss, S., Meyer, D. S., Klebba, I., Balwierz, P. J., van Nimwegen, E., and Bentires-Alj, M. (2013). Parity induces differentiation and reduces Wnt/Notch signaling ratio and proliferation potential of basal stem/progenitor cells isolated from mouse mammary epithelium. Breast cancer research: BCR 15, R36.

Mukhopadhyay, C., Zhao, X., Maroni, D., Band, V., and Naramura, M. (2013). Distinct effects of EGFR ligands on human mammary epithelial cell differentiation. PloS one 8, e75907.

Neve, R. M., Chin, K., Fridlyand, J., Yeh, J., Baehner, F. L., Fevr, T., Clark, L., Bayani, N., Coppe, J. P., Tong, F., et al. (2006). A collection of breast cancer cell lines for the study of functionally distinct cancer subtypes. Cancer cell 10, 515-527.

Pan, D. (2010). The hippo signaling pathway in development and cancer. Developmental cell 19, 491-505.

Perrimon, N., Pitsouli, C., and Shilo, B. Z. (2012). Signaling mechanisms controlling cell fate and embryonic patterning. Cold Spring Harbor perspectives in biology 4, a005975.

Petersen, O. W., and Polyak, K. (2010). Stem cells in the human breast. Cold Spring Harbor perspectives in biology 2, a003160.

Pilat, M. J., Christman, J. K., and Brooks, S. C. (1996). Characterization of the estrogen receptor transfected MCF10A breast cell line 139B6. Breast cancer research and treatment 37, 253-266.

Pobbati, A. V., and Hong, W. (2013). Emerging roles of TEAD transcription factors and its coactivators in cancers. Cancer biology & therapy 14, 390-398.

Regad, T., Bellodi, C., Nicotera, P., and Salomoni, P. (2009). The tumor suppressor Pml regulates cell fate in the developing neocortex. Nature neuroscience 12, 132-140.

Regan, J. L., Kendrick, H., Magnay, F. A., Vafaizadeh, V., Groner, B., and Smalley, M. J. (2012). c-Kit is required for growth and survival of the cells of origin of Brca1-mutation-associated breast cancer. Oncogene 31, 869-883.

Rios, A. C., Fu, N. Y., Lindeman, G. J., and Visvader, J. E. (2014). In situ identification of bipotent stem cells in the mammary gland. Nature 506, 322-327.

Sanchez Alvarado, A., and Yamanaka, S. (2014). Rethinking differentiation: stem cells, regeneration, and plasticity. Cell 157, 110-119.

Santagata, S., Thakkar, A., Ergonul, A., Wang, B., Woo, T., Hu, R., Harrell, J. C., McNamara, G., Schwede, M., Culhane, A. C., et al. (2014). Taxonomy of breast cancer based on normal cell phenotype predicts outcome. The Journal of clinical investigation 124, 859-870.

Sarkar, A., and Hochedlinger, K. (2013). The sox family of transcription factors: versatile regulators of stem and progenitor cell fate. Cell stem cell 12, 15-30.

Shehata, M., Teschendorff, A., Sharp, G., Novcic, N., Russell, A., Avril, S., Prater, M., Eirew, P., Caldas, C., Watson, C. J., et al. (2012). Phenotypic and functional characterization of the luminal cell hierarchy of the mammary gland. Breast cancer research: BCR 14, R134.

Skibinski, A., Breindel, J. L., Prat, A., Galvan, P., Smith, E., Rolfs, A., Gupta, P. B., Labaer, J., and Kuperwasser, C. (2014). The Hippo transducer TAZ interacts with the SWI/SNF complex to regulate breast epithelial lineage commitment. Cell reports 6, 1059-1072.

Smart, C. E., Wronski, A., French, J. D., Edwards, S. L., Asselin-Labat, M. L., Waddell, N., Peters, K., Brewster, B. L., Brooks, K., Simpson, K., et al. (2011). Analysis of Brca1-deficient mouse mammary glands reveals reciprocal regulation of Brca1 and c-kit. Oncogene 30, 1597-1607.

Soule, H. D., Maloney, T. M., Wolman, S. R., Peterson, W. D., Jr., Brenz, R., McGrath, C. M., Russo, J., Pauley, R. J., Jones, R. F., and Brooks, S. C. (1990). Isolation and characterization of a spontaneously immortalized human breast epithelial cell line, MCF-10. Cancer research 50, 6075-6086.

Spike, B. T., Engle, D. D., Lin, J. C., Cheung, S. K., La, J., and Wahl, G. M. (2012). A mammary stem cell population identified and characterized in late embryogenesis reveals similarities to human breast cancer. Cell stem cell 10, 183-197.

Stampfer, M., Hallowes, R. C., and Hackett, A. J. (1980). Growth of normal human mammary cells in culture. In vitro 16, 415-425.

Stingl, J. (2011). Estrogen and progesterone in normal mammary gland development and in cancer. Hormones & cancer 2, 85-90.

Stingl, J., Emerman, J. T., and Eaves, C. J. (2005). Enzymatic dissociation and culture of normal human mammary tissue to detect progenitor activity. Methods in molecular biology 290, 249-263.

Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.

Sun, P., Yuan, Y., Li, A., Li, B., and Dai, X. (2010). Cytokeratin expression during mouse embryonic and early postnatal mammary gland development. Histochemistry and cell biology 133, 213-221.

Takahashi, Y., Miyoshi, Y., Takahata, C., Irahara, N., Taguchi, T., Tamaki, Y., and Noguchi, S. (2005). Down-regulation of LATS1 and LATS2 mRNA expression by promoter hypermethylation and its association with biologically aggressive phenotype in human breast cancers. Clinical cancer research: an official journal of the American Association for Cancer Research 11, 1380-1385.

Tanaka, E. M., and Reddien, P. W. (2011). The cellular basis for animal regeneration. Developmental cell 21, 172-185.

Tata, P. R., Mou, H., Pardo-Saganta, A., Zhao, R., Prabhu, M., Law, B. M., Vinarsky, V., Cho, J. L., Breton, S., Sahay, A., et al. (2013). Dedifferentiation of committed epithelial cells into stem cells in vivo. Nature 503, 218-223.

Tong, Q., and Hotamisligil, G. S. (2007). Developmental biology: cell fate in the mammary gland. Nature 445, 724-726.

Tschaharganeh, D. F., Xue, W., Calvisi, D. F., Evert, M., Michurina, T. V., Dow, L. E., Banito, A., Katz, S. F., Kastenhuber, E. R., Weissmueller, S., et al. (2014). p53-Dependent Nestin Regulation Links Tumor Suppression to Cellular Plasticity in Liver Cancer. Cell 158, 579-592.

van Es, J. H., Sato, T., van de Wetering, M., Lyubimova, A., Nee, A. N., Gregorieff, A., Sasaki, N., Zeinstra, L., van den Born, M., Korving, J., et al. (2012). DII1+ secretory progenitor cells revert to stem cells upon crypt damage. Nature cell biology 14, 1099-1104.

Van Keymeulen, A., Rocha, A. S., Ousset, M., Beck, B., Bouvencourt, G., Rock, J., Sharma, N., Dekoninck, S., and Blanpain, C. (2011). Distinct stem cells contribute to mammary gland development and maintenance. Nature 479, 189-193.

Varelas, X. (2014). The Hippo pathway effectors TAZ and YAP in development, homeostasis and disease. Development 141, 1614-1626.

Villadsen, R., Fridriksdottir, A. J., Ronnov-Jessen, L., Gudjonsson, T., Rank, F., LaBarge, M. A., Bissell, M. J., and Petersen, O. W. (2007). Evidence for a stem cell hierarchy in the adult human breast. The Journal of cell biology 177, 87-101.

Visvader, J. E., and Lindeman, G. J. (2006). Mammary stem cells and mammopoiesis. Cancer research 66, 9798-9801.

Visvader, J. E., and Stingl, J. (2014). Mammary stem cells and the differentiation hierarchy: current status and perspectives. Genes & development 28, 1143-1158.

Yang, N., Morrison, C. D., Liu, P., Miecznikowski, J., Bshara, W., Han, S., Zhu, Q., Omilian, A. R., Li, X., and Zhang, J. (2012). TAZ induces growth factor-independent proliferation through activation of EGFR ligand amphiregulin. Cell cycle 11, 2922-2930.

Yeh, W. L., Shioda, K., Coser, K. R., Rivizzigno, D., McSweeney, K. R., and Shioda, T. (2013). Fulvestrant-induced cell death and proteasomal degradation of estrogen receptor alpha protein in MCF-7 cells require the CSK c-Src tyrosine kinase. PloS one 8, e60889.

Yimlamai, D., Christodoulou, C., Galli, G. G., Yanger, K., Pepe-Mooney, B., Gurung, B., Shrestha, K., Cahan, P., Stanger, B. Z., and Camargo, F. D. (2014). Hippo pathway activity influences liver cell fate. Cell 157, 1324-1338.

Zhang, H., Pasolli, H. A., and Fuchs, E. (2011). Yes-associated protein (YAP) transcriptional coactivator functions in balancing growth and differentiation in skin. Proc Natl Acad Sci USA 108, 2270-2275.

Zhang, J., Ji, J. Y., Yu, M., Overholtzer, M., Smolen, G. A., Wang, R., Brugge, J. S., Dyson, N. J., and Haber, D. A. (2009). YAP-dependent induction of amphiregulin identifies a non-cell-autonomous component of the Hippo pathway. Nature cell biology 11, 1444-1450.

Zhang, J., Smolen, G. A., and Haber, D. A. (2008). Negative regulation of YAP by LATS1 underscores evolutionary conservation of the Drosophila Hippo pathway. Cancer research 68, 2789-2794.

Zhao, B., Li, L., Lei, Q., and Guan, K. L. (2010). The Hippo-YAP pathway in organ size control and tumorigenesis: an updated version. Genes & development 24, 862-874.

Zhao, B., Li, L., Wang, L., Wang, C. Y., Yu, J., and Guan, K. L. (2012). Cell detachment activates the Hippo pathway via cytoskeleton reorganization to induce anoikis. Genes & development 26, 54-68.

Zhou, W., and Slingerland, J. M. (2014). Links between oestrogen receptor activation and proteolysis: relevance to hormone-regulated cancer therapy. Nature reviews Cancer 14, 26-38.

1. A method for treating breast cancer in a subject having a breast cancer of the estrogen receptor (ERα) negative type, wherein the method comprises the step of administering to said subject a therapeutically effective amount of a modulator of the Large Tumor Suppressor Kinase (LATS).

2. The method of claim 1, wherein said modulator inhibits the interaction between the estrogen receptor α and LATS.

3. The method of claim 2, wherein said inhibitor does not affect the kinase activity of LATS.

4. The method of any of claim 1, wherein said method further comprises the step of administering to said subject a therapeutically effective amount of an antagonist of the estrogen receptor after the step of administering to said subject a therapeutically effective amount of a modulator of the Large Tumor Suppressor Kinase (LATS).

5. The method of any of claim 4 wherein said antagonist of the estrogen receptor is not fulvestrant.

6. The method of claim 1 wherein the modulator is administered to the subject before, during or after radiation therapy, chemotherapy targeted therapy or after surgical removal of a primary tumor.

7. The method of any of claim 1, wherein the cancer is a solid tumor.

8. The method of claim 1, wherein said modulator is an antibody or a siRNA.

9. The method of claim 8, wherein the siRNA decreases or silences the expression of the Large Tumor Suppressor Kinase (LATS).

10. The method of claim 1, wherein the modulator is an antibody that specifically binds to the Large Tumor Suppressor Kinase (LATS).

11. The method of claim 10, wherein said antibody inhibits the interaction between the estrogen receptor 1 and LATS.

12. A combination for use to treat breast cancer of the estrogen receptor (ER) negative type, which combination comprises (a) a modulator of the Large Tumor Suppressor Kinase (LATS) and (b) an antagonist of the estrogen receptor, wherein the active ingredients are present in each case in free form or in the form of a pharmaceutically acceptable salt or any hydrate thereof, and optionally at least one pharmaceutically acceptable carrier; for simultaneous, separate or sequential use, wherein in case of sequential use, the a modulator of the Large Tumor Suppressor Kinase is administered first.

13. The combination according to claim 12 wherein the modulator of the Large Tumor Suppressor Kinase (LATS) inhibits the interaction between the estrogen receptor α and LATS.

14. The combination according to claim 12 wherein the modulator of the Large Tumor Suppressor Kinase (LATS) is a siRNA and the antagonist of the estrogen receptor is tamoxifen.
